(19) United States
(12) Patent Application Publication
(10) Pub. No.: US 2024/0148256 A1
(43) Pub. Date: May 9, 2024

(54) CIRCULATING BLOOD VOLUME DETERMINATION APPARATUS, CIRCULATING BLOOD VOLUME DETERMINATION PROGRAM, AND CIRCULATING BLOOD VOLUME DETERMINATION METHOD

(71) Applicant: NIHON KOHDEN CORPORATION, Tokyo (JP)

(72) Inventors: Naoki KOBAYASHI, Tokorozawa-shi, Saitama (JP); Haruka MORIMOTO, Tokorozawa-shi, Saitama (JP); Taiki GOTO, Tokorozawa-shi, Saitama (JP)

(73) Assignee: NIHON KOHDEN CORPORATION, Tokyo (JP)

(21) Appl. No.: 18/282,742

(22) PCT Filed: May 18, 2022
(86) PCT No.: PCT/JP2022/020690

§ 371 Date: Sep. 18, 2023

(30) Foreign Application Priority Data
May 19, 2021 (JP) 2021-084561

(51) Int. Cl. A61B 5/02; A61B 5/00; A61B 5/021; A61B 5/022

(52) U.S. Cl. CPC A61B 5/02028; A61B 5/02108; A61B 5/022; A61B 5/6824; A61B 2562/0247

(57) ABSTRACT

The presently disclosed subject matter provides a circulating blood volume determination apparatus includes a detection unit configured to detect a pulse wave from a sensor attached to a predetermined portion of a living body, an analysis unit configured to analyze a state of a venous component in the detected pulse wave, and a determination unit configured to perform a determination as to a circulating blood volume based on the analysis result of the analysis unit.

### TECHNICAL FIELD

The presently disclosed subject matter relates to a circulating blood volume determination apparatus, a circulating blood volume determination program, and a circulating blood volume determination method.

### BACKGROUND ART

Maintaining a circulating blood volume in a human body is important for sufficient supply of oxygen to entire organs. For example, when the circulating blood volume in the human body decreases due to dehydration or the like, sufficient blood is not supplied to the organs, which may cause renal failure, myocardial ischemia, cerebrovascular accidents or the like. In particular, dehydration is relatively likely to occur on preoperative subjects because of fasting before surgery. Further, vasodilation due to general anesthesia lowers a blood pressure. A decrease in the blood pressure also causes a lack of blood flow to the organs. Therefore, fluid infusion may be required after induction of anesthesia.

On the other hand, in recent years, it has been pointed out that, with regard to the fluid infusion, since the circulating blood volume excessively increases due to excessive fluid infusion, the organs in the whole body is brought into an edema state, and there is a possibility of causing disorders such as pulmonary complications, acute kidney injuries, sepsis, and wound healing delay.

Accordingly, both the excessive circulating blood volume and the insufficient circulating blood volume may cause disorders or the like.

Indexes used in fluid infusion management include pulse pressure variation (PPV), systolic pressure variation (SPV), and stroke volume variation (SVV). However, in order to monitor these indexes, it is necessary to insert an intravascular catheter, so that there is a problem of being highly invasive and taking time and effort for medical workers.

A technique in the related art is disclosed in JP-A-2015-160082 (Patent Literature 1) below. A pulse wave component is extracted from a pressure waveform measured by maintaining a cuff pressure applied by a cuff attached to an upper arm of a subject at a constant pressure equal to or lower than a diastolic blood pressure. A respiratory cycle of the subject is calculated based on a change in amplitude of the pulse wave component. A respiratory variation of an arterial pressure of the subject is calculated based on a variation in amplitude of the pulse wave component in a plurality of respiratory cycles. Further, the cuff pressure is decreased stepwise. A cuff pressure when the amplitude of the pulse wave component shows a peak is determined as an average venous pressure. Then, when the respiratory variation of the arterial pressure is lower than a threshold value and the average venous pressure is higher than a threshold value, it is determined that a circulating blood volume is excessive. Further, when the respiratory variation of the arterial pressure exceeds the threshold value and the average venous pressure falls below the threshold value, it is determined that the circulating blood volume is insufficient.

### SUMMARY OF INVENTION

### Technical Problem

On the other hand, in order to make the circulating blood volume appropriate, it is desired to accurately determine the stop or start of the fluid infusion in a short time. There is a problem that the technique in the related art cannot sufficiently meet such a demand.

The presently disclosed subject matter has been made to solve the above-described problems. That is, an object is to provide a circulating blood volume determination apparatus, a circulating blood volume determination program, and a circulating blood volume determination method capable of accurately determining in a short time whether procedure for making a circulating blood volume appropriate is necessary.

### Solution to Problem

The above problems of the presently disclosed subject matter are solved by the following means.

(1) A circulating blood volume determination apparatus includes a detection unit configured to detect a pulse wave from a sensor attached to a predetermined portion of a living body, an analysis unit configured to analyze a state of a venous component in the detected pulse wave, and a determination unit configured to perform a determination as to a circulating blood volume based on the analysis result of the analysis unit.

(2) A circulating blood volume determination program causing a computer to execute a detection procedure of detecting a pulse wave from a sensor attached to a predetermined portion of a living body, an analysis procedure of analyzing a state of a venous component in the detected pulse wave, and a determination procedure of performing a determination as to a circulating blood volume based on the analysis result in the analysis procedure.

(3) A circulating blood volume determination method includes a detection step of detecting a pulse wave from a sensor attached to a predetermined portion of a living body, an analysis step of analyzing a state of a venous component in the detected pulse wave, and a determination step of performing a determination as to a circulating blood volume based on the analysis result in the analysis step.

### Advantageous Effects of Invention

The state of the venous component in the detected pulse wave is analyzed, and the determination as to the circulating blood volume is performed based on the analysis result. Accordingly, whether procedure for making the circulating blood volume appropriate is necessary can be accurately determined in a short time.

### BRIEF DESCRIPTION OF DRAWINGS

FIG. 1 is a block diagram illustrating a schematic hardware configuration of a circulating blood volume determination system.

FIG. 2 is a block diagram illustrating a schematic hardware configuration of a control unit.

FIG. 3 is a block diagram illustrating functions of the control unit.

FIG. 4 is a diagram illustrating cuff pressures and pulse waves when a circulating blood volume is excessive, insufficient, and appropriate.

FIG. 5 is a flow chart illustrating an operation of the circulating blood volume determination system.

FIG. 6 is a flow chart illustrating an operation of the circulating blood volume determination system.

### DESCRIPTION OF EMBODIMENTS

Hereinafter, embodiments of the presently disclosed subject matter will be described with reference to the accompanying drawings. In the drawings, the same members are denoted by the same reference numerals. Further, dimensional ratios in the drawings are exaggerated for convenience of description, and may be different from actual ratios.

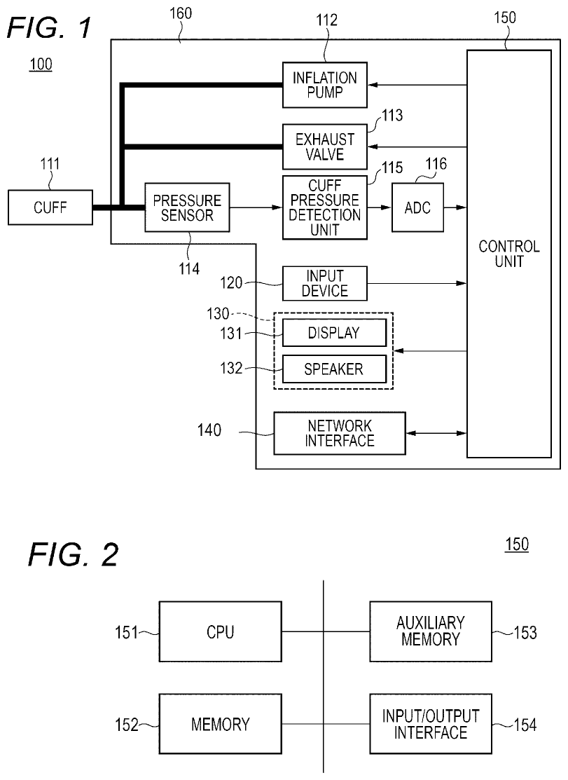

FIG. 1 is a block diagram illustrating a schematic hardware configuration of a circulating blood volume determination system 100 according to the present embodiment. FIG. 2 is a block diagram illustrating a schematic hardware configuration of a control unit 150.

As illustrated in the example of FIG. 1, the circulating blood volume determination system 100 can include a cuff 111 and a circulating blood volume determination apparatus 160. The cuff 111 is configured to be connected to the circulating blood volume determination apparatus 160. The circulating blood volume determination apparatus 160 can include an inflation pump 112, an exhaust valve 113, a pressure sensor 114, a cuff pressure detection unit 115, an AD converter (ADC) 116, an input device 120, an output device 130, a network interface 140, and the control unit 150. The output device 130 is implemented as an output unit.

The cuff 111, the inflation pump 112, the exhaust valve 113, the pressure sensor 114, the cuff pressure detection unit 115, and the ADC 116 are an example of a configuration that performs sensing processing of measuring a pulse wave of a subject, and are implemented as a sensor.

The cuff 111 is an air bladder wound around an upper arm of the subject. The upper arm of the subject is an example of a predetermined portion of a living body to which a sensor for detecting a pulse wave is attached. The inflation pump 112 sends air to the cuff 111 and increases a pressure in the air bladder (hereinafter referred to as “cuff internal pressure”) according to an instruction from the control unit 150. Accordingly, a pressing pressure (hereinafter referred to as “cuff pressure”) applied by the cuff 111 to the upper arm of the subject can be increased. The exhaust valve 113 opens the air in the air bladder to the atmosphere, and thus gradually exhausts the air from the cuff 111 to decrease the cuff internal pressure. Accordingly, the cuff pressure can be decreased.

The pressure sensor 114 detects the cuff internal pressure. A pulse wave of the subject (hereinafter, simply referred to as a “pulse wave”) is superimposed on the cuff internal pressure. The pulse wave is a pressure wave in which a venous pulse wave and an arterial pulse wave are superimposed. The cuff pressure detection unit 115 extracts the pulse wave superimposed on the cuff internal pressure from the detected cuff internal pressure, and outputs the cuff internal pressure and the extracted pulse wave as analog signals to the ADC 116. The ADC 116 converts the analog signals of the cuff internal pressure and the pulse wave into digital signals at a predetermined sampling frequency and transmits the digital signals to the control unit 150. The pressure sensor 114, the cuff pressure detection unit 115, and the ADC 116 may be integrated with the cuff 111. The pressure sensor 114 and the cuff pressure detection unit 115 may be integrated with the cuff 111, and the ADC 116 may be provided in the circulating blood volume determination apparatus 160. Alternatively, the ADC 116 may be integrated with the cuff 111, and the digital signals may be supplied to the circulating blood volume determination apparatus 160.

The input device 120 receives an input operation performed by a user who operates the circulating blood volume determination apparatus 160, and generates an input signal corresponding to the input operation. The input device 120 includes, for example, a touch panel disposed to overlap a display 131 of the output device 130, an operation button, a mouse, a keyboard, or the like attached to a case of the circulating blood volume determination apparatus 160. The input signal generated by the input device 120 is transmitted to the control unit 150. The control unit 150 performs predetermined processing according to the input signal. As to be described later, the input operation includes an input of a predetermined condition for a state of a venous component in the pulse wave.

The output device 130 outputs a determination result for the circulating blood volume. The determination result includes at least one of determination results including that the circulating blood volume of the subject is excessive, appropriate, or insufficient (or any index capable of estimating that the circulating blood volume of the subject is excessive, appropriate, or insufficient). The output device 130 includes the display 131 and a speaker 132. The display 131 may be a liquid crystal display, an organic EL display, or the like attached to the case of the circulating blood volume determination apparatus 160. The display 131 may be a display device such as a transmissive or non-transmissive head-mounted display mounted on a head of the user.

The speaker 132 is attached to the case of the circulating blood volume determination apparatus 160, and can output the determination result of the circulating blood volume by voice.

The output device 130 is not limited to the display 131 and the speaker 132, and may be, for example, a printer for printing and outputting the determination result for the circulating blood volume.

The network interface 140 is configured to connect the control unit 150 to a communication network. Specifically, the network interface 140 includes processing circuits for various interfaces for communicating with an external device via a communication network, and is configured to conform to a communication standard for communicating via the communication network. The communication network is a local area network (LAN), a wide area network (WAN), the Internet, or the like.

The control unit 150 is software and hardware that mainly controls the circulating blood volume determination apparatus 160 and executes various calculations, and the control unit 150 may be an independent device. For example, the control unit 150 may be a dedicated medical device for testing circulating blood volume, or may be a personal computer, a smartphone, a tablet terminal, or the like in which a program for testing the circulating blood volume is installed. Further, the control unit 150 may be a wearable device or the like attached to a body (for example, the upper arm or the like) of the subject.

Details of the functions of the control unit 150 will be described later.

As illustrated in FIG. 2, the control unit 150 can include a central processing unit (CPU) 151, a memory 152, an auxiliary memory 153, and an input/output interface 154.

The memory 152 can include a read only memory (ROM) and a random access memory (RAM). Various programs, various parameters, and the like are stored in the ROM. Further, the RAM can include a work area in which various programs to be executed by the CPU 151 are stored. The CPU 151 is configured to load, on the RAM, a program specified from various programs stored in the ROM or the auxiliary memory 153 as a non-transitory computer-readable storage medium and execute various processes in cooperation with the RAM.

The auxiliary memory 153 can include, for example, a storage device (storage) such as a hard disk drive (HDD), a solid state drive (SSD), or a USB flash memory. The auxiliary memory 153 stores various programs and various types of data. Further, the auxiliary memory 153 stores the determination result for the circulating blood volume.

The input/output interface 154 functions as an interface between the CPU 151 and the input device 120 and the output device 130. The input/output interface 154 includes various communication modules that communicate with the input devices such as the mouse and the keyboard, and a drive module that drives the display 131 and the speaker 132, or the like.

When the CPU 151 executes the program, the control unit 150 controls respective units of the circulating blood volume determination system 100 to implement various functions.

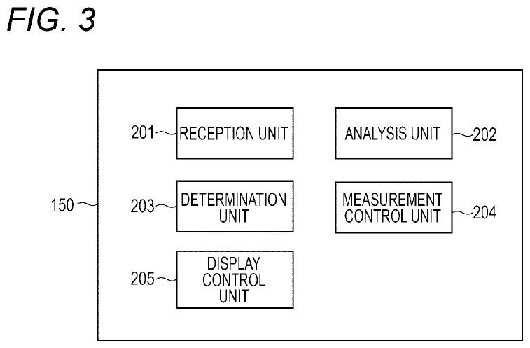

FIG. 3 is a block diagram illustrating the functions of the control unit 150. The control unit 150 functions as a reception unit 201, an analysis unit 202, a determination unit 203, a measurement control unit 204, and a display control unit 205. The measurement control unit 204 is implemented as a venous pressure calculation unit and a pulse wave variation calculation unit.

The reception unit 201 receives the predetermined condition for the state of the venous component in the pulse wave input by the input device 120. The predetermined condition is a condition for determining whether the circulating blood volume is excessive. The state of the venous component in the pulse wave includes, for example, at least one of a shape of the venous pulse wave, a ratio of the venous pulse wave, and magnitude of the venous pulse wave. The predetermined condition may be, for example, that a predetermined percentile of the pulse wave is equal to or greater than a predetermined threshold value (second threshold value). The predetermined percentile of the pulse wave corresponds to the shape of the venous pulse wave. The predetermined condition may be that the ratio of a pulse wave of the venous pulse wave extracted from the pulse wave by a known method to the pulse wave is equal to or greater than a predetermined threshold value. The predetermined condition may be that an amplitude of the venous pulse wave extracted from the pulse wave is equal to or greater than a predetermined threshold value. The fact that the amplitude of the venous pulse wave extracted from the pulse wave is equal to or greater than the predetermined threshold value includes that the venous pulse wave is detected from the pulse wave. That is, the predetermined threshold value of the amplitude of the venous pulse wave may be a minimum value among values at which it is considered that presence of the venous pulse wave is recognized to be meaningful. Each threshold value can be appropriately set by experiment from the viewpoint of accuracy of the determination result for the circulating blood volume. The predetermined condition may be a condition for a predetermined statistical index value of the pulse wave. The statistical index value includes, for example, at least one of an average pressure value, a maximum pressure value, a minimum pressure value, a sum of squares, RMS, a variance, a gradient, a kurtosis, and a skewness. These statistical index values correspond to the shape of the venous pulse wave. For example, when the predetermined condition is a condition for the average pressure value, the predetermined condition may be that the average pressure value is equal to or greater than a predetermined threshold value. For the sake of simplicity, the following description will be made on the assumption that the predetermined condition is that the predetermined percentile of the pulse wave is less than the predetermined threshold value.

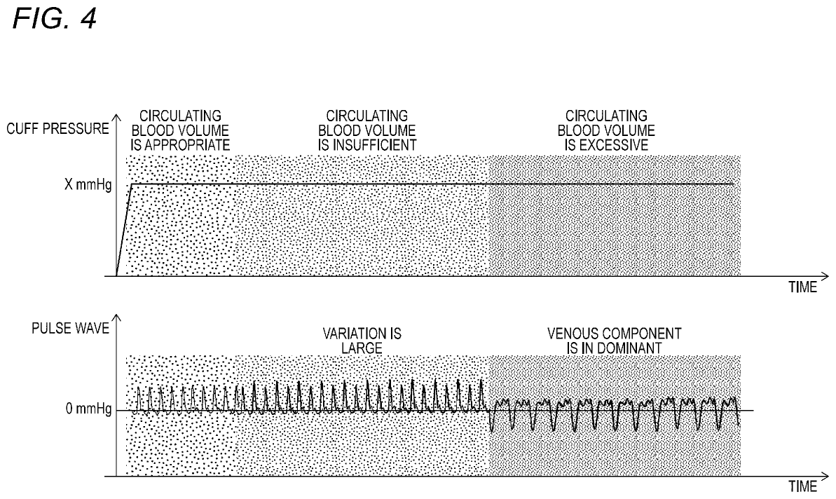

FIG. 4 is a diagram illustrating the cuff pressures and the pulse waves when the circulating blood volume is excessive, insufficient, and appropriate. In FIG. 4, in order to clarify that each pulse wave is detected in a state where the cuff pressure is constant and same (predetermined pressure to described later), for convenience, the pulse waves when the circulating blood volume is excessive, insufficient, and appropriate is shown in one diagram.

The pulse wave of the subject whose circulating blood volume is excessive has a characteristic shape (waveform) in which a negative component is larger than that of the pulse wave of the subject whose circulating blood volume is appropriate and that of the pulse wave of the subject whose circulating blood volume is insufficient. Therefore, by setting a predetermined percentile at which the circulating blood volume be distinguished by the difference in shape of the pulse wave, it can be determined that the circulating blood volume is excessive when the predetermined percentile of the pulse wave is less than the predetermined threshold value. When the predetermined percentile of the pulse wave is equal to or greater than the predetermined threshold value, it can be determined that the circulating blood volume is appropriate or insufficient.

The amplitude (a difference between a top and a bottom) of the pulse wave of the subject whose circulating blood volume is insufficient significantly varies as compared with that of the pulse wave of the subject whose circulating blood volume is appropriate. Therefore, whether the circulating blood volume is insufficient or appropriate can be distinguished based on magnitude of the variation of the amplitude of the pulse wave. The magnitude of the variation of the amplitude of the pulse wave corresponds to a pulse pressure variation (hereinafter referred to as “PPV”) of an artery to be described later.

The analysis unit 202 analyzes the state of the venous component in the pulse wave for the pulse wave detected by the measurement control unit 204. Specifically, the analysis unit 202 analyzes the state of the venous component in the pulse wave by dividing the detected pulse wave into certain intervals and calculating a predetermined percentile of the pulse wave in each interval. The predetermined percentile is, for example, 50 percentile or less. For example, 25 percentile is a value of digital data at a position of 25% from a piece of head digital data when the digital data (sampling data) of a waveform of the pulse wave in one cycle (one heartbeat) after the AD conversion is performed thereon by the ADC 116 is arranged in ascending order of the sampling value (mmHg). The digital data of the waveform in one cycle may be an average value of digital data of a plurality of waveforms corresponding to each heartbeat included in the pulse wave. The 25 percentile may be calculated from a pulse wave in a certain interval (for example, a pulse wave for 10 seconds).

The determination unit 203 performs a determination as to the circulating blood volume based on the analysis result of the analysis unit 202. Specifically, the determination unit 203 determines whether the predetermined percentile of the pulse wave calculated by the analysis unit 202 is less than the predetermined threshold value. Then, when it is determined that the predetermined percentile of the pulse wave is less than the predetermined threshold value (when it is determined that the predetermined condition is satisfied), the determination unit 203 determines that the circulating blood volume is excessive. When it is determined that the predetermined percentile of the pulse wave is equal to or greater than the predetermined threshold value (when it is determined that the predetermined condition is not satisfied), the determination unit 203 determines that the circulating blood volume is appropriate or insufficient.

As described above, the state of the venous component in the pulse wave includes, for example, at least one of the shape of the venous pulse wave, the ratio of the venous pulse wave, and the magnitude of the venous pulse wave. Therefore, instead of the processing using the predetermined percentile, the determination unit 203 may perform a determination as to the circulating blood volume based on similarity between a measured shape of the pulse wave and the typical shapes of the pulse wave when the circulating blood volume is appropriate, insufficient, or excessive. Further, the state of the venous component in the pulse wave may be two or more states among the shape of the venous pulse wave, the ratio of the venous pulse wave, and the magnitude of the venous pulse wave.

The measurement control unit (detection unit) 204 sets the cuff pressure to a predetermined pressure by increasing the cuff internal pressure of the cuff 111, and detects (measures) the pulse wave by the pressure sensor 114 and the cuff pressure detection unit 115 in a state where the cuff pressure is maintained at the predetermined pressure. The predetermined pressure may be determined in advance as the cuff pressure at which the venous pulse wave can be detected. For example, the predetermined pressure may be freely determined with reference to a target venous pressure (for example, 8 mmHg). The measurement control unit may detect the pulse wave while the cuff pressure is increasing to the predetermined pressure.

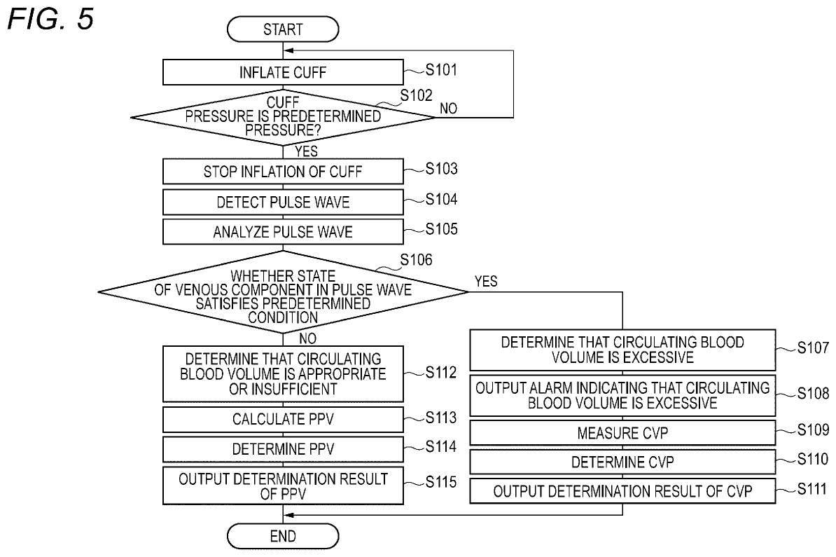

The measurement control unit 204 may perform only the processing of FIG. 5 to be described later, or may perform the processing of FIG. 5 in the process of measuring a systolic blood pressure or a diastolic blood pressure.

When the determination unit 203 determines that the circulating blood volume is excessive, the measurement control unit 204 can calculate the venous pressure such as a center venous pressure (hereinafter referred to as “CVP”) by using a known method such as an oscillometric method by further decreasing or increasing the cuff internal pressure. That is, for example, the cuff internal pressure is increased by a predetermined increase rate, and the pulse wave is detected by the pressure sensor 114 and the cuff pressure detection unit 115. The detection of the pulse wave performed together with the increase of cuff internal pressure is repeatedly performed until the amplitude of the pulse wave changes by a predetermined amount. Then, the cuff internal pressure at the time when the amplitude of the pulse wave is maximum is estimated as the venous pressure. The reason why the cuff internal pressure when the amplitude of the pulse wave is maximum can be estimated as the venous pressure is that the amplitude of the pulse wave is maximum when the cuff internal pressure is equal to the venous pressure.

When the determination unit 203 determines that the circulating blood volume is appropriate or insufficient, the measurement control unit 204 may calculate the PPV (pulse pressure variation of the artery) based on the detected pulse wave. That is, the PPV is calculated by a known method based on a pulse wave of only an arterial component (hereinafter, also referred to as an “arterial pulse wave”) obtained in a state where a venous blood vessel is closed. PPV is a value obtained by dividing a difference between a maximum amplitude and a minimum amplitude of the arterial pulse wave in one respiration by an average of the maximum amplitude and the minimum amplitude of the arterial pulse wave, and represents a variation rate of the amplitude of the pulse wave in one respiration.

The determination unit 203 can further determine whether the CVP and the PPV calculated by the measurement control unit 204 are abnormal. Specifically, when the CVP exceeds an upper limit of a predetermined normal range, the determination unit 203 determines that the CVP is abnormal for being high. When the CVP is less than a lower limit of the normal range, the determination unit 203 determines that the CVP is abnormal for being low. When the CVP is included in the normal range, it is determined that the CVP is normal. The determination unit 203 determines that the circulating blood volume is insufficient based on a comparison between the PPV and a predetermined threshold value (first threshold value). Specifically, when the PPV exceeds the predetermined threshold value, the determination unit 203 determines that the PPV is abnormal. When the PPV is equal to or less than the predetermined threshold value, the determination unit 203 determines that the PPV is normal.

The display control unit 205 displays, on the display 131, the determination result of the determination unit 203. Specifically, the display control unit 205 displays whether the circulating blood volume is excessive or the circulating blood volume is appropriate or insufficient. When the circulating blood volume is excessive or insufficient (when the circulating blood volume is not normal), the display control unit 205 can display/voice-output the determination result as an alarm. Accordingly, for example, during surgery or the like, it is possible to promptly notify that the fluid infusion needs to be adjusted in order to make the circulating blood volume appropriate.

When it is determined that the CVP is abnormal, the display control unit (output unit) 205 displays the determination result together with the CVP value as an alarm. When it is determined that the PPV is abnormal, the display control unit 205 displays (outputs) the determination result together with the PPV value as an alarm. When the CVP is normal and when the PPV is normal, the display control unit 205 can display the determination result together with each measurement value.

Instead of displaying the determination result on the display 131, such as displaying that the circulating blood volume is excessive as an alarm, the display control unit 205 may voice-output the determination result from the speaker 132 or output (transmit) the determination result to an external terminal or the like via the network interface 140.

FIG. 5 is a flowchart illustrating an operation of the circulating blood volume determination system 100. The present flowchart can be executed by the control unit 150 according to a program stored in the auxiliary memory 153.

The control unit 150 increases the cuff pressure (S101), and determines whether the cuff pressure has reached a predetermined pressure (S102). When it is determined that the cuff pressure has not reached the predetermined pressure (S102: NO), the control unit 150 executes step S101 and step S102 until the cuff pressure reaches the predetermined pressure.

When it is determined that the cuff pressure has reached the predetermined pressure (S102: YES), the control unit 150 stops increasing the cuff pressure (S103). Accordingly, the cuff pressure is maintained at the predetermined pressure.

The control unit 150 detects the pulse wave by the pressure sensor 114, the cuff pressure detection unit 115, or the like (S104).

The control unit 150 analyzes the state of the venous component in the pulse wave for the detected pulse wave (S105).

The control unit 150 determines whether the state of the venous component in the pulse wave satisfies a predetermined condition (S106).

When it is determined that the state of the venous component in the pulse wave satisfies the predetermined condition (S106: YES), the control unit 150 determines that the circulating blood volume is excessive (S107). Then, the control unit 150 outputs an alarm indicating that the circulating blood volume is excessive (S108). Further, the control unit 150 measures the CVP (S109). The control unit 150 determines whether the CVP is abnormal (S110). Then, the control unit 150 outputs the determination result of the CVP (S111).

When it is determined that the state of the venous component in the pulse wave does not satisfy the predetermined condition (S106: NO), the control unit 150 determines that the circulating blood volume is appropriate or insufficient (S112). The control unit 150 increases the cuff pressure, and calculates the PPV based on the pulse wave detected while maintaining the cuff pressure in a range in which the pulse wave does not include the venous pulse wave, and which is equal to or less than the diastolic blood pressure (S113). The control unit 150 determines whether the PPV is abnormal (S114). Then, the control unit 150 outputs the determination result of the PPV (S115). The processing in S109 and S113 may not be performed.

### Second Embodiment

A second embodiment will be described. In the first embodiment, the cuff 111 or the like is used as an example of a sensor that detects the pulse wave. Meanwhile, in the present embodiment, a photoelectric volume pulse wave sensor is attached to a predetermined portion of a living body as a sensor to detect a pulse wave. Since the present embodiment is the same as or similar to the first embodiment in other points, redundant description will be omitted or simplified.

The predetermined portion to which the photoelectric volume pulse wave sensor is attached is a portion in which the state of the venous component in the pulse wave satisfies the predetermined condition only when the circulating blood volume is excessive. That is, the predetermined portion is a portion in which the state of the venous component in the pulse wave satisfies the predetermined condition when the circulating blood volume is excessive, but does not satisfy the predetermined condition when the circulating blood volume is appropriate or insufficient. The predetermined portion may be determined in advance by an experiment or the like.

Hereinafter, the functions of the control unit 150 will be described with reference to FIG. 3 again.

The reception unit 201 receives the predetermined condition for the state of the venous component in the pulse wave input by the input device 120. The predetermined condition is, for example, that a predetermined percentile of the pulse wave is equal to or greater than a predetermined threshold value.

The analysis unit 202 analyzes the state of the venous component in the pulse wave detected by the measurement control unit 204 using the photoelectric volume pulse wave sensor (not shown) attached to the predetermined portion of the subject. Specifically, for example, the analysis unit 202 calculates a predetermined percentile of the pulse wave. The predetermined portion is not limited as long as the state of the venous component in the pulse wave satisfies the predetermined condition only when the circulating blood volume is excessive. The predetermined portion may be, for example, a fingertip. Instead of the photoelectric volume pulse wave sensor, another sensor capable of detecting the pulse wave may be used.

The determination unit 203 performs a determination the circulating blood volume based on the analysis result of the analysis unit 202. Specifically, for example, the determination unit 203 determines whether the predetermined percentile of the pulse wave calculated by the analysis unit 202 is less than a predetermined threshold value. Then, when it is determined that the predetermined percentile of the pulse wave is less than the predetermined threshold value (when it is determined that the predetermined condition is satisfied), the determination unit 203 determines that the circulating blood volume is excessive. When it is determined that the predetermined percentile of the pulse wave is equal to or greater than the predetermined threshold value (when it is determined that the predetermined condition is not satisfied), the determination unit 203 determines that the circulating blood volume is appropriate or insufficient.

The measurement control unit 204 detects the pulse wave by driving the photoelectric volume pulse wave sensor attached to the predetermined portion of the subject.

The display control unit 205 displays, on the display 131, the determination result of the determination unit 203. Specifically, the display control unit 205 displays whether the circulating blood volume is excessive or the circulating blood volume is appropriate or insufficient. Specifically, when the circulating blood volume is excessive, the display control unit 205 can display the determination result as an alarm. When the circulating blood volume is appropriate or insufficient, the display control unit 205 can display the determination result. When the circulating blood volume is excessive, a display prompting the measurement of CVP may be displayed. When the circulating blood volume is appropriate or insufficient, a display prompting measurement of the PPV may be displayed.

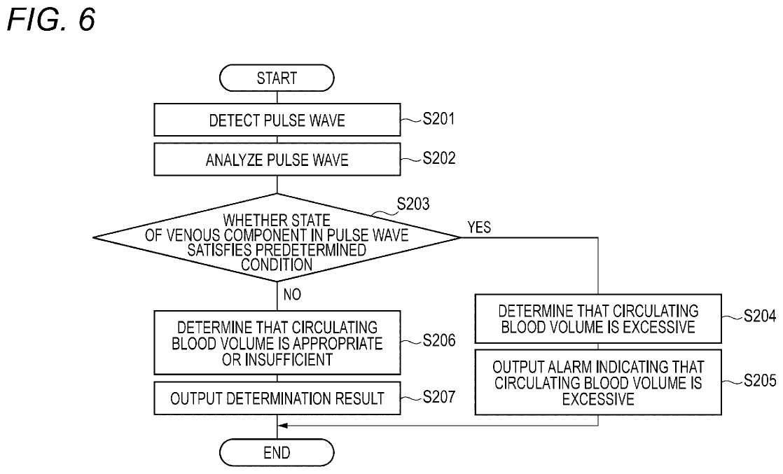

FIG. 6 is a flowchart illustrating an operation of the circulating blood volume determination system 100.

The control unit 150 detects a pulse wave by the photoelectric volume pulse wave sensor (S201).

The control unit 150 analyzes a state of an venous component in the pulse wave for the detected pulse wave (S202).

The control unit 150 determines whether the state of the venous component in the pulse wave satisfies a predetermined condition (S203).

When it is determined that the state of the venous component in the pulse wave satisfies the predetermined condition (S203: YES), the control unit 150 determines that the circulating blood volume is excessive (S204). Then, the control unit 150 outputs an alarm indicating that the circulating blood volume is excessive (S205).

When it is determined that the state of the venous component in the pulse wave does not satisfy the predetermined condition (S203: NO), the control unit 150 determines that the circulating blood volume is appropriate or insufficient (S206). Then, the control unit 150 outputs the determination result that the circulating blood volume is appropriate or insufficient (S207).

The embodiments have the following effects.

The state of the venous component in the detected pulse wave is analyzed, and the determination as to circulating blood volume is performed based on the analysis result. Accordingly, whether procedure for making the circulating blood volume appropriate is necessary can be accurately determined in a short time.

Further, when it is determined that the state of the venous component satisfies the predetermined condition, it is determined that the circulating blood volume is excessive. Accordingly, the necessity of stopping the fluid infusion can be accurately determined in a short time.

Further, when it is determined that the state of the venous component does not satisfy the predetermined condition, it is determined that the circulating blood volume is appropriate or insufficient. Accordingly, it is possible to determine in a short time that fluid infusion may be needed.

Further, a cuff is used as a sensor for detecting the pulse wave, and the pulse wave is detected based on a pressure received by the cuff from a predetermined portion when the predetermined portion is pressurized by the cuff at a predetermined pressure. Accordingly, whether procedure for making the circulating blood volume appropriate is necessary can be more easily and accurately determined in a short time. The CVP and the PPV can be quickly measured as necessary while avoiding complications such as sensor replacement.

Further, the pulse wave is detected in the process of increasing the cuff pressure, and when it is determined that the state of the venous component satisfies the predetermined condition based on the analysis result of the state of the venous component in the pulse wave, the venous pressure is calculated based on the detected pulse wave, and is output together with the determination result. Accordingly, for example, when a systolic blood pressure and a diastolic blood pressure are measured by depressurization measurement, whether the procedure for making the circulating blood volume appropriate is necessary can be determined at the same time.

Further, when it is determined that the state of the venous component satisfies the predetermined condition, an alarm for warning that the circulating blood volume is excessive is output. Accordingly, when it is necessary to stop the fluid infusion, a medical worker can be notified of the necessity.

Further, when it is determined that the state of the venous component does not satisfy the predetermined condition, the pulse wave variation is calculated based on the pulse wave, and it is determined that the circulating blood volume is insufficient according to a comparison between the pulse wave variation and the predetermined first threshold value. Accordingly, the necessity of the fluid infusion can be accurately determined in a short time.

Further, the predetermined condition for determining whether the circulating blood volume is excessive is that the predetermined percentile of the pulse wave is equal to or greater than the predetermined second threshold value. Accordingly, whether procedure for making the circulating blood volume appropriate is necessary can be more accurately determined.

Further, the predetermined condition is a condition for a predetermined statistical index value of the pulse wave. Accordingly, whether procedure for making the circulating blood volume appropriate is necessary can be more accurately determined.

Further, when it is determined that the circulating blood volume is insufficient, an alarm for warning that the circulating blood volume is insufficient is output. Accordingly, when the fluid infusion is necessary, the medical worker can be notified of the necessity.

Although the embodiments of the present invention have been described in detail above, the present invention is not limited to the above-described embodiments.

For example, when a degree of similarity between a template of the pulse wave in a case where the circulating blood volume is excessive and a detected pulse wave is equal to or greater than a predetermined threshold value, the control unit may determine that the predetermined condition is satisfied (the circulating blood volume is excessive). When the degree of similarity between the template and the pulse wave is less than the predetermined threshold value, it may be determined that the predetermined condition is not satisfied (the circulating blood volume is appropriate or insufficient).

The control unit may determine (estimate) whether the predetermined condition is satisfied (the circulating blood volume is excessive) based on the detected pulse wave using a machine learning model trained in advance. A trained model that is trained by using a relatively large amount of known training data in which a pulse wave when the circulating blood volume is excessive is used as input data and data indicating that a predetermined condition is satisfied (for example, “1”) is used as a correct label may be used as the machine learning model.

The control unit may determine that the circulating blood volume is insufficient when a degree of similarity between a template of the pulse wave in a case where the circulating blood volume is insufficient and a detected pulse wave is equal to or greater than a predetermined threshold value.

A part of or all of the functions implemented by the program in the above-described embodiments may be implemented by hardware such as a circuit.

In the flowchart described above, a part of the steps may be omitted, or other steps may be added. A part of the steps may be executed at the same time, or one step may be divided into a plurality of steps and executed.

This application claims priority to Japanese Patent Application No. 2021-084561 filed on May 19, 2021, the entire content of which is incorporated herein by reference.

1. A circulating blood volume determination apparatus comprising:
a detection unit configured to detect a pulse wave from a sensor attached to a predetermined portion of a living body;
an analysis unit configured to analyze a state of a venous component in the detected pulse wave; and
a determination unit configured to perform a determination as to a circulating blood volume based on the analysis result of the analysis unit.

2. The circulating blood volume determination apparatus according to claim 1, wherein
the determination unit determines that the circulating blood volume is excessive when it is determined that the state of the venous component satisfies a predetermined condition based on the analysis result of the analysis unit.

3. The circulating blood volume determination apparatus according to claim 2, wherein
the determination unit determines that the circulating blood volume is appropriate or insufficient when it is determined that the state of the venous component does not satisfy the predetermined condition based on the analysis result of the analysis unit.

4. The circulating blood volume determination apparatus according to claim 1, wherein
the sensor is a cuff, and the detection unit detects the pulse wave based on a pressure received by the cuff from a predetermined portion while the predetermined portion is pressurized by the cuff at a predetermined pressure.

5. The circulating blood volume determination apparatus according to claim 4, wherein
the detection unit detects the pulse wave in a process of increasing, to the predetermined pressure, a pressure at which the predetermined portion is pressurized by the cuff, the process of increasing the pressure including a process of maintaining the pressure at the predetermined pressure,
the circulating blood volume determination apparatus further comprises:
a venous pressure calculation unit configured to calculate a venous pressure based on the detected pulse wave when the determination unit determines that the state of the venous component satisfies the predetermined condition based on the analysis result of the analysis unit; and
an output unit configured to output the calculated venous pressure together with the determination result of the determination unit.

6. The circulating blood volume determination apparatus according to claim 5, wherein
the output unit further outputs an alarm warning that the circulating blood volume is excessive when the determination unit determines that the state of the venous component satisfies the predetermined condition.

7. The circulating blood volume determination apparatus according to claim 3, further comprising:
a pulse wave variation calculation unit configured to calculate a pulse wave variation based on the detected pulse wave when the determination unit determines that the state of the venous component does not satisfy the predetermined condition; and
an output unit configured to output the calculated pulse wave variation together with the determination result of the determination unit, wherein
the determination unit determines that the circulating blood volume is insufficient according to a comparison between the calculated pulse wave variation and a predetermined first threshold value.

8. The circulating blood volume determination apparatus according to claim 2, wherein
the predetermined condition is that a predetermined percentile of the pulse wave is less than a predetermined second threshold value, and
the analysis unit analyzes the state of the venous component in the pulse wave by calculating the predetermined percentile of the pulse wave.

9. The circulating blood volume determination apparatus according to claim 2, wherein
the predetermined condition is a condition for a predetermined statistical index value of the pulse wave.

10. The circulating blood volume determination apparatus according to claim 7, wherein
the output unit further outputs an alarm warning that the circulating blood volume is insufficient when it is determined that the circulating blood volume is insufficient.

11. A non-transitory computer-readable storage medium that stores a circulating blood volume determination program causing a computer to execute procedures of:
a detection procedure of detecting a pulse wave from a sensor attached to a predetermined portion of a living body;
an analysis procedure of analyzing a state of a venous component in the detected pulse wave; and
a determination procedure of performing a determination as to a circulating blood volume based on the analysis result in the analysis procedure.

12. A circulating blood volume determination method comprising:
a detection step of detecting a pulse wave from a sensor attached to a predetermined portion of a living body;
an analysis step of analyzing a state of a venous component in the detected pulse wave; and
a determination step of performing a determination as to a circulating blood volume based on the analysis result in the analysis step.
